(19) United States
(12) Patent Application Publication
(10) Pub. No.: US 2011/0004172 A1
(43) Pub. Date: Jan. 6, 2011

(54) Device for vacuum therapy of wounds

(75) Inventors: Axel Eckstein, Heidenheim (DE); Ulrich Fink, Heidenheim (DE); Pierre Croizat, Herbrechtingen (DE)

Correspondence Address: DREISS, FUHLENDORF, STEIMLE & BECKER, POSTFACH 10 37 62, D-70032 STUTTGART, DE

(73) Assignee: Paul Hartmann AG, Heidenheim (DE)

(21) Appl. No.: 12/801,266

(22) Filed: Jun. 1, 2010

Related U.S. Application Data
(60) Provisional application No. 61/295,183, filed on Jan. 15, 2010.

(30) Foreign Application Priority Data
Jul. 6, 2009 (DE) 10 2009 031 992.1

(51) Int. Cl. A61M 1/00; A61L 15/46

(52) U.S. Cl. 604/313

(57) ABSTRACT

Device for vacuum therapy of wounds comprising a cover material (1) that is impermeable to air, and a means for functional connection of the wound space (4) with a source of vacuum outside the cover material, and at least one activated absorbent/rinsing body (3) that contains at least one superabsorbent polymer.

This application claims benefit to 61/295,183 filed Jan. 15, 2010 as well as Paris Convention priority to DE 10 2009 031 992.1 filed Jul. 6, 2009 the entire disclosures of which are hereby incorporated by reference.

### BACKGROUND OF THE INVENTION

The invention relates to a new device for vacuum therapy of wounds comprising a cover material that is impermeable to air for air-tight closure of the wound and the wound environment, and a means for functional connection of the wound space with a source of vacuum outside the cover material so that a vacuum can be established in the wound space and liquids can be sucked out of the wound space.

A wound is defined as the severing of the connection of tissues of the integumentary system of human beings and animals. It can be associated with a loss of substance.

Devices for the vacuum therapy of wounds are known according to the prior art.

For example, WO1993/009727 describes a device to promote wound healing by the application of a vacuum to the region of the skin containing and surrounding the wound. The device according to WO1993/009727 comprises a vacuum means for producing the vacuum, an air-tight covering of the wound termed the sealing means, which is functionally connected to the vacuum means, and a wound dressing termed the screen structure for positioning on the wound within the sealing means. The screen structure is an open-cell polymer foam, for example, polyester foam. As can be seen from the description of WO1993/009727, application of the vacuum therapy can accelerate the wound healing of different types of wounds, for example, burns, pressure sores, or contused wounds.

During the treatment, the vacuum can be continuously maintained until the dressing is changed. Alternatively, the vacuum can be applied during alternating cycles, or cycles with different levels of vacuum can be applied.

Devices for vacuum therapy of wounds are commercially available, for example, the V.A.C.® device from KCI. In commercially available devices, a wound dressing is often deployed that contains an open-cell polymer foam, such as polyvinylalcohol (PVA) or polyurethane (PU).

Foam dressings are compressed to different degrees depending on the vacuum applied. This can result in a narrowing of the passages necessary for removal of the wound exudate. Furthermore, in a long application of polyvinylalcohol or polyurethane foam in vacuum therapy, the foam can adhere to the wound base. Adhering foam must be removed with great effort when the dressing is changed, for example, by rinsing with Ringer's solution. Tissue that has grown into the foam can result in tissue traumatization when the dressing is changed and impair wound healing.

In addition to the use of open-cell polymer foam, other materials have been described for the production of wound dressings for the vacuum therapy of wounds. WO2001/89431 describes a collagen matrix as a wound dressing for the vacuum therapy of wounds.

GB2415908 describes the use of fibrous material that can also be bioresorbable in wound dressings for the vacuum therapy of wounds.

WO2006/52839 describes the use of dry fibrous materials or a fibrous mixture of materials as a wound contact layer for the vacuum therapy of wounds. During the vacuum therapy, the fibrous material forms a gel by the absorption of wound exudate. A foam layer may be interposed between the wound contact layer formed from the gel and the cover material. The foam layer can replace the cover material and perform the function of an air-tight cover material.

In an embodiment of WO 2006/52839, the wound can be rinsed. This is achieved by a tube additionally introduced into the wound space, through which a rinsing liquid can be pumped into the wound. The rinsing liquid is removed from the wound space through the tube that is also used to produce and maintain the vacuum. A liquid trap must be connected upstream of the pump producing the vacuum. The rinsing device requires a complicated assembly that is only possible in stationary equipment.

WO2005/123170 discloses wound dressings for vacuum therapy by means of which unwanted substances are deactivated or removed from the wound space and/or desired substances present in the wound space are concentrated. As components of wound dressings that are suitable for the desired purpose, WO2005/123170 proposes numerous initially dry polymers that can form gels by the absorption of water from the wound exudate. The gel-forming polymers permit the elimination of a liquid trap between the dressing and the pump because the wound exudate is retained in the wound exudate and does not enter the suction tube. As suitable polymers, for example, transversely cross-linked polyacrylate gels and superabsorbent gels are proposed.

WO2006/048246 describes a multi-component dressing for vacuum therapy of wounds, which contains a superabsorbent polymer. The superabsorbent polymer can be held within a liquid-tight envelope and form a dry absorbent pad that is placed in the wound space. During the vacuum therapy, the volume of the absorbent pad expands as it absorbs liquid from the wound exudate.

WO2006/056294 and WO2006/048240 also describe drainage means that contain a superabsorbent polymer for the vacuum therapy of wounds. Between the absorbent pad and the wound space, a protective element can be interposed that is non-irritant to the mucous membrane. During the vacuum therapy, the cross-sectional area of the absorbent pad greatly expands and approximates to a circular shape. As can be seen from WO2006/056294, expansion of the initial volume of the absorbent pad is expressly desired in order to retain the wound secretions thus absorbed in the absorbent pad until the absorbent pad is removed from the wound space. The inventive large increase in volume is enabled by the application of a dry absorbent body into the wound.

The object of this invention is to improve the vacuum therapy of wounds still further and to overcome the disadvantages of the prior art. In particular, the object of this invention is to provide devices and methods for the vacuum therapy of wounds with which treatment can be performed as effectively and gently as possible.

### SUMMARY OF THE INVENTION

The invention solves the object with a device for the vacuum therapy of wounds, an air-impermeable cover material for air-tight closure of the wound and the wound environment, a means for the functional connection of the wound space to a source of vacuum located outside the cover material so that a vacuum can be established in the wound space and liquids can be sucked out of the wound space and at least one activated absorbent/rinsing body that contains at least one superabsorbent polymer for insertion in the interstice formed between the wound surface and the cover material. The object of the invention is also solved with an activated absorbent/rinsing body for use in the vacuum therapy of wounds, the activated absorbent/rinsing body containing at least one superabsorbent polymer.

Surprisingly, it has been discovered that a device for vacuum therapy of wounds comprising a cover material that is impermeable to air for the air-tight closure of the wound and the wound environment, a means for the functional connection of the wound space with a vacuum source located outside the cover material so that a vacuum can be applied in the wound space and liquids can be sucked out of the wound space, and at least one activated absorbent/rinsing body that contains at least one superabsorbent polymer for inclusion in the interstice that is formed between the wound surface and the cover material, is suitable for the very effective and very gentle treatment of wounds.

The new inventive device is characterized, in particular, by that fact that it comprises at least one activated absorbent/rinsing body that contains at least one superabsorbent polymer.

A “superabsorbent polymer” is generally understood to be a water-insoluble, swellable polymer that can absorb a multiple of its own weight of liquid such as water, saline solutions, or body fluids. The absorption of liquid results in the formation of a hydrogel. The absorption capacity for pure water is typically greater than the absorption capacity for saline liquid. In connection with this invention, the term “superabsorbent polymer” refers, in particular, to a polymer exhibiting a w value (free swell capacity) according to the standard test method WSP 240.2 (05) of at least 10 g/g, preferably at least 20 g/g. The test method WSP 240.2 (05) for determining the w value is described in “Standard Test Methods for the Nonwovens and Related Industries,” 2008 edition (published by “EDANA, International Association Serving the Nonwovens and Related Industries,” Cary, N.C., U.S.A. and “INDA, Association of the Nonwovens Fabrics Industry,” Brussels, Belgium).

WSP 240.2 (05) according to EDANA is a standard test method for determining the w value (free swell capacity) of superabsorbent polyacrylate powder. According to WSP 240.2 (05), the free absorption capacity for a 0.9 percent by weight common salt solution is determined.

In connection with this invention, determination of the w value of superabsorbent material that is not polyacrylate powder is performed in a similar way.

The superabsorbent polymer can preferably be provided in the form of particles or fibers.

According to the invention, an activated absorbent/rinsing body is understood to be an absorbent/rinsing body that contains a superabsorbent polymer and that has been activated by means of an aqueous activation solution. “Aqueous activation solution” is understood to be a liquid with a water content of at least 50 percent by weight. As a consequence of the activation, the activated absorbent/rinsing body contains at least 500 percent by weight of an aqueous activation solution in relation to the weight of the dry absorbent/rinsing body. The at least one superabsorbent polymer contained in an activated absorbent/rinsing body is at least partially in a swollen condition as a result of the activation.

In a further embodiment, the activated absorbent/rinsing body contains at least 600 percent by weight, preferably at least 800 percent by weight, especially preferably at least 1000 percent by weight of an aqueous activation solution, all in relation to the weight of the dry absorbent/rinsing body.

Furthermore, the activated absorbent/rinsing body preferably contains less than 5000 percent by weight, in particular, preferably less than 3500 percent by weight, especially preferably less than 2500 percent by weight of an aqueous activation solution.

In an especially preferred embodiment, the activated absorbent/rinsing body contains the quantity of an aqueous activation solution that corresponds to its maximum absorption capacity for Ringer's solution. The maximum absorption capacity for Ringer's solution can be determined by the above-stated test method WSP 240.2 (05), wherein however

a) Ringer's solution is used instead of the common salt solution used in WSP 240.2 (05) and

b) the inventive absorbent/rinsing body is used instead of a test substance sealed in an envelope (“bag” according to paragraph 6.1 of WSP 240.2 (05)).

The maximum absorption capacity corresponds to the weight difference determined by gravimetric means according to this method between the dry absorbent/rinsing body and the activated absorbent/rinsing body, including an upward or downward deviation of the weight difference by 15 percent.

The activated absorbent/rinsing body contains at least one superabsorbent polymer and, if applicable, one or more auxiliary or substrate substances. It can be surrounded by an envelope that permits the passage of liquids at least in certain regions.

The inventive absorbent/rinsing body differs from dry absorbent pads known from vacuum therapy prior art in that it is an activated absorbent/rinsing body. Surprisingly, advantageous characteristics in the vacuum therapy of wounds can be achieved by the activation of the absorbent/rinsing body.

One advantage of the activated absorbent/rinsing body as a component of the inventive device is that it provides a soft wound dressing for the vacuum therapy of wounds.

A further advantage of the activated absorbent/rinsing body as a component of the inventive device is that even distribution of pressure over the base of the wound can be ensured.

By the provision of a soft wound dressing and by the even distribution of pressure, vacuum therapy can be performed gently and especially effectively.

A further advantage of the activated absorbent/rinsing body as a component of the inventive device is that adhering and/or growing together of the wound base with the wound dressing can largely be avoided. This is advantageous because no additional cleaning of the wound is required after removal of the vacuum dressing. Dressing changing can be performed more gently and faster. Traumatization of the wound during dressing changing can be avoided. This increases the effectiveness of the wound therapy.

A further advantage of the activated absorbent/rinsing body as a component of the inventive device is that a hydroactive effect that supports healing arises immediately after commencement of the vacuum therapy. A hydroactive effect in this case is understood to be that the wound is kept in a moist environment and does not dry out. As has already been known for a long time (see, for example, G D Winter (1962) in Nature 193, pp. 293-294), drying out of the wound results in scurfiness, which can occur very quickly, for example, within 24 hours. The scurfiness slows down wound healing. The hydroactive effect, on the other hand, suppresses the scurfiness and promotes the growth of the granulation tissue. A hydroactive effect beginning early on can therefore very favorably influence the progress of wound healing.

The hydroactive effect achieved by using the inventive activated absorbent/rinsing body can be explained by the observation that the activated absorbent/rinsing body can release an activation solution during vacuum therapy. As a consequence of the release of the activation solution, the activated absorbent/rinsing body is also able to absorb wound exudate in exchange. In particular, the activated absorbent/rinsing body is able to absorb bacteria and harmful components of the wound exudate. Absorption of bacteria and harmful components of the wound exudate additionally promotes wound healing and counteracts wound infections.

In particular, an advantageous rinsing effect can be achieved by the activated absorbent/rinsing body during vacuum therapy. This permits the cleaning of the wound during therapy and is advantageous for wound healing. The rinsing effect is especially advantageous during the so-called cleansing phase at the beginning of the wound healing process. A marked rinsing effect can, in particular, be achieved if varying levels of vacuum are applied.

In a preferred embodiment, the invention provides a device for the vacuum therapy of wounds in which the superabsorbent polymer is a particle or fiber.

In a further preferred embodiment, the invention provides a device for the vacuum therapy of wounds in which the superabsorbent polymer is a superabsorbent polyacrylate.

For the purpose of this invention, the polyacrylate is understood to be a synthetic polymer, comprising acrylic acid (2-propenoic acid, CH₂═CH—CO₂H) and/or a salt thereof, as a monomer (M1). The monomer component is, in particular, more than 70 percent by weight acrylic acid and/or a salt thereof (in relation to the total weight of the polyacrylate). Inventive polyacrylates preferably contain a monomer component of more than 80 percent by weight acrylic acid and/or a salt thereof and highly preferably more than 95 percent by weight acrylic acid and/or a salt thereof in relation to the total weight of the polyacrylate. The polyacrylate can be present as a homopolymer, copolymer, or block polymer. If the polyacrylate is present as a copolymer or block polymer, the monomer component of the monomer M1 in the polymer is in any case more than 70 percent by weight, in particular, more than 80 percent by weight and highly preferably more than 95 percent by weight in relation to the total weight of the polyacrylate. In these copolymer polyacrylates or block-polymer polyacrylates, in addition to the monomer M1, in particular, α,β-unsaturated ether (vinyl ether), α,β-unsaturated carboxylic acids, or α,β-unsaturated carboxylic acid esters (vinyl esters) can be contained as comonomers M2. Among the comonomers M2 of the α,β-unsaturated carboxylic acids, in particular, methacrylic acid(2-methylpropenoic acid), ethacrylic acid(2-ethylpropenoic acid), crotonic acid(2-butenoic acid), sorbic acid(trans-trans-2,4-hexadienoic acid), maleic acid(cis-2-butenedioic acid) or fumaric acid(trans-2-butenedioic acid) are preferred. However, the polyacrylate may also consist of a) a homopolymer of acrylic acid and/or b) a copolymer of i) acrylic acid and a salt of acrylic acid, ii) of methacrylic acid and a salt of the methacrylic acid or iii) of acrylic acid and methacrylic acid and its salts. However, the polyacrylate may also be a mixture of different polyacrylates.

Here, in particular, the α,β-unsaturated carboxylic acids and the acrylic acids may be present in a neutralized form as salt, in a non-neutralized form as free acid or in mixtures of the two. In particular, polyacrylates that are based on acrylic acid and salts of acrylic acid have proven especially suitable. Among these, alkaline metal or alkaline-earth metal. salts are especially noteworthy. In particular, polyacrylates consisting of homopolymers and/or copolymers that include monomer acrylic acid and/or sodium or potassium acrylate have proven suitable especially for the inventive absorbent/rinsing body.

Moreover, it has been shown that polyacrylates from the group of cross-linked and/or transversely cross-linked and/or surface-cross-linked polyacrylates are especially suitable. These polyacrylates preferably comprise a) a homopolymer that consists of the monomers M1 and is cross-linked and/or transversely cross-linked by means of a cross-linking agent, and/or b) a copolymer that consists of the monomers M1 and M3, wherein the monomer M1 is acrylic acid and/or a salt thereof and the monomer M3 is chosen from the group of the cross-linking agents. This means that these polyacrylates comprise a polyacrylate that has been subsequently cross-linked by means of a cross-linking agent and/or a polyacrylate that has been copolymerized from acrylic acid and/or a salt thereof and a cross-linking agent. In particular, it has been shown that cross-linked and/or transversely cross-linked polyacrylates that, as cross-linking agents, contain V1 compounds with at least two ethylenically unsaturated groups within one molecule or compounds V2 that contain at least two functional groups that can react with functional groups of the acrylic acid and/or a salt thereof in a condensation reaction, in an addition reaction, or in a ring-opening reaction, or compounds V3 that contain at least one ethylenically unsaturated group and at least one functional group that can react with functional groups of the acrylic acid and/or of a salt thereof and/or the α,β-unsaturated comonomers in a condensation reaction, in an addition reaction, or in a ring-opening reaction, are especially suitable for the inventive absorbent/rinsing body. By means of the V1 compounds, in particular, a cross-linking of the polymers is achieved by the radical polymerization of the ethylenically unsaturated groups of the cross-linking agent molecule with the monoethylenically unsaturated monomer acrylic acid and/or of one of its salts and/or of one of the α,β-unsaturated comonomers, while in the case of compounds V2, cross-linking of the polymers is achieved by condensation reaction of the functional groups with the functional groups of the acrylic acid and/or of one of its salts or of one of the α,β-unsaturated comonomers. In the case of the compounds V3, cross-linking of the polymer is therefore achieved both by radical polymerization of the ethylenically unsaturated group and by condensation reaction between the functional group of the cross-linking agent and the functional groups of the monomers. Preferred V1 compounds are polyacrylic acid esters or polymethacrylic acid esters that, for example, are obtained by the reaction of a polyol, for example, ethylene glycol(ethane-1,2-diol), propylene glycol(propane-1,2-diol), trimethylolpropane (2-(hydroxymethyl)-2-ethylpropane-1,3-diol), 1,6-hexandiol, glycerol(propane-1,2,3-triol), pentaerythritol(2,2-Bis(hydroxymethyl)1,3-propanediol), polyethylene glycol (HO—(CH₂—CH₂—O)ₙ—H where n=2 to 20) where n=1 to 20), polypropylene glycol (HO—(CH(CH₃)—CH₂—O)ₙ—H where n=2 to 20), of an amino alcohol, of a polyalkylene polyamine, such as, for example, diethylentriamine or triethylentetriamine, or of an alcoxylated polyol with acrylic acid or methacryl acid. The cross-linked polyacrylate can preferably be a polyacrylate that is cross-linked by means of a V1 compound that is a di-, tri-, or tetraester of the polyacrylic acid or polymethacrylic acid that is obtained by the reaction of an alcoxylated polyol, in particular, of an ethoxylated polyol, in particular, ethoxylated ethylene glycol, ethoxylated propylene glycol, ethoxylated trimethylolpropane, ethoxylated 1,6-hexandiol or ethoxylated glycerine, with an average number of ethylene oxide units n per hydroxy group of n=1 to 10 with acrylic acid or methacrylic acid. As V1 compounds, furthermore, polyvinyl compounds, polyallyl compounds, polymethylallyl compounds, acrylic acid esters or methacrylic acid esters of a monovinyl compound, acrylic acid esters or methacrylic acid esters of a monoallyl compound or monomethylallyl compound, preferably of the monoallyl compounds or monomethallyl compounds of a polyol or of an amino alcohol are preferred. In this connection, reference is made to DE 195 43 366 and DE 195 43 368.

The superabsorbent polymers of polyacrylate stated in the invention are characterized by an excellent absorption capacity and the ability to exchange liquids and are therefore especially suitable for the inventive device. The polymers further permit a rinsing effect because the affinity of the polyacrylate with the protein-containing wound exudate is greater than the affinity with the salt-containing activation solution (for example, Ringer's solution). In this way, the wound exudate will displace the activation solution from the wound pad.

Further advantages of the superabsorber described above are a soft material structure, good physiological compatibility, low toxicity, and a high level of safety.

In a preferred embodiment, the superabsorbent polymer is a mixture of polyacrylate particles, wherein the polyacrylate particles exhibit the chemical composition stated in connection with the invention. The particle mixture may also contain polyacrylate particles that differ with respect to the polyacrylates, that is, the particle mixture comprises at least two different kinds of polyacrylate particles.

The polyacrylates can differ, for example, in their degree of neutralization, their degree of cross-linking, in the cross-linking agent and/or the copolymers. In the simplest case, however, polyacrylate particles can also be used that are identical with respect to their structure, only the stated particle sizes being different. In particular, the polyacrylate particles of a first size range a) can differ from the polyacrylate particles of a second size range b).

However, the particles of the first or second particle size range can consist of different polyacrylate particles, that is, for example, the particles of the first size range comprise first polyacrylate particles and second the particles of the second size range comprise polyacrylate particles differing from the first polyacrylate particles, wherein both sorts comprise polyacrylate particles that are within their respective size ranges.

In connection with this invention, a particle mixture is to be understood as a mixture whose individual components (particles) can be spatially adjacent, partially mixed, completely mixed, or spatially separate, wherein the mixture is in any case to be seen as a component of an absorbent/rinsing body. In particular, the particles of the individual particle size ranges can be spatially adjacent, partially mixed, completely mixed, or spatially separate.

The activated absorbent/rinsing body thus comprises a particle mixture with a defined quantity of a first particle fraction with a defined particle size and a second quantity of a second particle fraction with a second particle size that is different from the first particle size, wherein each particle fraction contains polyacrylate particles. The polyacrylate particles of one size range may comprise particles of the same or different sizes, wherein the particle size(s) is/are within the range. In connection with this invention, data stating the quantity of particles, in particular, of an individual particle size range always refer to a percentage by weight in relation to the totality of particles, unless stated otherwise.

The particle size of the dry particles is determined in connection with this invention by analogy with standard test WSP 220-2-(05), wherein the sieves (diameter 200 mm) have hole sizes in accordance with the specifications given. The standard test WSP 220-2-(05) is described in “Standard Test Methods for the Nonwovens and Related Industries,” 2008 edition. Moreover, sieves with other hole sizes, such as 125 μm, 160 μm, 630 μm, 900 μm, and 1500 μm can be used. This is based on dry polyacrylate particles with a moisture content of less than 5 percent by weight of water in relation to the total weight of the particles, wherein the moisture content is determined according to WSP 230.02 (05). The standard test WSP 230-2-(05) is described in “Standard Test Methods for the Nonwovens and Related Industries,” 2008 edition.

The particle size should be determined according to the method stated above before the absorbent/rinsing body containing the particles is manufactured. It is not possible to determine the particle size on an already activated absorbent/rinsing body because the test method WSP 230-2-(05) refers to particles with a moisture content of less than 5 percent by weight.

It is known from WO2009/68249 that polyacrylate particles both inhibit and compartmentalize proteases by direct binding and can therefore draw them out of the wound exudate or the wound. An excess, in particular, of metalloproteases is undesirable, in particular, in the granulation phase of wound healing. In one embodiment of the inventive device that contains an activated absorbent/rinsing body with superabsorbent polyacrylate, the polyacrylate particles intercept and/or inhibit an excess of metalloproteases in chronic wounds in such a way that healing can progress naturally. For this purpose, as can be seen from WO2009/68249, polyacrylate particles with a particle size x, where x≦300 μm, are especially suitable. Particles of this size especially effectively inhibit the activity of proteases that are harmful to wound healing, in particular, metalloproteases in a wound. Polyacrylate particles with a particle size x, where x≦300 μm, are however less well or not at all suitable for absorbing wound exudate because these particles absorb and retain a very much smaller quantity of aqueous liquids than particles with a particle size x, where x>300 μm. It is therefore advantageous if the activated absorbent/rinsing body contains a mixture of polyacrylate particles including both particles that, because of their size, can inhibit and compartmentalize proteases and particles that, because of their size, exhibit a great absorption capacity.

In an especially preferred embodiment of the inventive device for the vacuum therapy of wounds, the superabsorbent polymer is a particle mixture of polyacrylate particles of different sizes, characterized in that the particle mixture contains

a) 5 to 100 percent by weight of particles with a particle size x, where x≦300 μm, and

b) 0 to 95 percent by weight of particles with a particle size x, where x>300 μm

In particular, the superabsorbent polymer is a particle mixture of polyacrylate particles of different sizes, characterized in that the particle mixture contains

a) 5 to 98 percent by weight of particles with a particle size x, where x≦300 μm, as a means of inhibiting proteases in the wound and

b) 2 to 95 percent by weight of particles with a particle size x, where x>300 μm, as a means for the relinquishment and/or release of aqueous solutions.

The superabsorbent polymer is therefore highly preferably a particle mixture of polyacrylate particles of different sizes, characterized in that the particle mixture contains

a) 20 to 80 percent by weight with a particle size x, where ≦300 μm, as a means of inhibiting proteases in the wound and

b) 20 to 80 percent by weight of particles with a particle size x, where x>300 μm, as a means for the relinquishment and/or release of aqueous solutions.

The activated absorbent/rinsing body can include in addition to the superabsorbent polymer, which is preferably a superabsorbent polyacrylate, a substrate material, the substrate material comprising a hydrophilic fiber material.

The hydrophilic material used in this case can be, in particular, water-insoluble fibers of cellulose, in particular, largely delignified technical cellulose fibers, in particular, wood fibers, in particular with a fiber length of <5 mm. The fiber material can also contain hydrophilic fiber material from regenerated cellulose, carboxymethyl cellulose, carboxyethyl cellulose, hydroxymethyl cellulose, or hydroxyethyl cellulose. A fiber mixture comprising cellulose, regenerated cellulose, carboxymethyl cellulose, carboxyethyl cellulose, hydroxymethyl cellulose, or hydroxyethyl cellulose fibers and fibers of polyethylene, polypropylene, or polyester may also be used.

In an especially preferred embodiment, the activated absorbent/rinsing body comprises at least one superabsorbent polyacrylate of the composition stated above and as the substrate material a mixture of cellulose fibers and polypropylene fibers.

These fibers can be processed in a so-called air-laid method together with the particle mixture to form one layer.

In an especially preferred embodiment, the activated absorbent/rinsing body is an enclosed air-laid that comprises a superabsorbent polyacrylate, cellulose and polypropylene fibers.

In this especially preferred embodiment, the air-laid can be enveloped in a textile material, in particular, the envelope can consist of an interlock knitted fabric made of polypropylene fibers. An air-laid enveloped in polypropylene that contains superabsorbent polyacrylate, cellulose, and polypropylene fibers is commercially available under the name TenderWet® (Paul Hartmann AG, Germany).

An absorbent/rinsing body that is plastically deformable and especially suitable for the inventive device is in particular obtained if its envelope at least partially comprises a textile surface that is non-elastically stretchable in the longitudinal, transverse, and diagonal directions. In this connection, reference is made to EP0594034B1.

One of several suitable methods for welding envelopes of thermoplastic materials is ultrasonic welding. The activated absorbent/rinsing body can, for example, have a round, rectangular, or oval shape. However, other shapes are also possible.

The activated absorbent/rinsing body can have different quantities of superabsorbent polymer and substrate. The activated absorbent/rinsing body preferably contains at least 10 percent by weight polyacrylate (in relation to the substrate material), wherein the polyacrylate has the composition stated in connection with the invention. However, activated absorbent/rinsing bodies are preferred that have at least 20 percent by weight, in particular, at least 25 percent by weight, and highly preferably at least 30 percent by weight polyacrylate (in relation to the substrate material). So as not to restrict the activated absorbent/rinsing body with respect to the relinquishment and release of aqueous liquids, it should be ensured, however, that the polyacrylate content in relation to the substrate material is preferably no more than 90 percent by weight and, in particular, no more than 75 percent by weight.

This invention provides a very effective and gentle device for vacuum therapy of wounds. To be able to achieve these advantages, it is essential that the device comprises an activated absorbent/rinsing body. It is therefore essential that the activated absorbent/rinsing body contain at least 500 percent by weight, in relation to the weight of the dry absorbent/rinsing body, of an aqueous activation solution.

However, it is especially advantageous if the activated absorbent/rinsing body contains at least 500 percent by weight, in relation to the weight of the dry absorbent/rinsing body, of an aqueous activation solution, comprising
more than 50 percent by volume water
at least 5 mmol/l sodium ions

with the specification that the activation solution is a synthetic activation solution.

In further preferred embodiments, the activated absorbent/rinsing body contains at least 600 percent by weight, preferably at least 800 percent by weight, especially preferably at least 1000 percent by weight in relation to the weight of the dry absorbent/rinsing body, of the synthetic activation solution stated above.

The activated absorbent/rinsing body preferably contains less than 5000 percent by weight, especially preferably less then 3500 percent by weight, highly preferably less than 2500 percent by weight of the synthetic activation solution.

In the context of this invention synthetic means that the activation solution is manufactured in a technical manner. Solutions that originate directly and without further processing from a living system such as a human being or animal are therefore not used as the activation solution. In particular, the activation solution does not consist of a solution that is secreted directly from a wound, that is, the activation solution is not human or animal wound exudate.

The activation solution can contain substances that are of biological origin on condition that these substances of the synthetic activation solution are added during manufacturing. Substances of biological origin are understood to be substances that are of human, animal, vegetable or microbial origin and/or are produced in such organisms (or parts thereof).

An absorbent/rinsing body activated with the synthetic activation solution has a soft structure and thus ensures even distribution of pressure. The synthetic activation solution permits the hydroactive effect and has a positive influence on the chemical environment of the wound.

Further, it has been found that by using an absorbent/rinsing body that contains at least 500 percent by weight in relation to the weight of the dry absorbent/rinsing body, of the aforementioned synthetic activation solution, adhesion and/or growing together of the wound dressing with the wound base can be reduced.

In a preferred embodiment, the activated absorbent/rinsing body contains at least 500 percent by weight, in relation to the weight of the dry absorbent/rinsing body, of a synthetic activation solution, comprising more than 50 percent by volume of water, at least 5 mmol/l of sodium ions, at least 0.1 mmol/l of potassium ions, at least 0.1 mmol/l of calcium ions and at least 5 mmol/l of chloride ions. The synthetic activation solution can optionally contain further inorganic cations and/or anions, possibly organic anions, possibly antimicrobial substances, and possibly added bioorganic compounds. The pH value is preferably 4 to 9. The viscosity at 20° C. is preferably between 0.8 mPa s and 150 mPa s. The viscosity of the activation solution was determined with a Brookfield viscometer (unit: 1 Pa·s=1 Ns/m²). The synthetic activation solution used in this preferred embodiment is especially suitable for activation of the absorbent/rinsing body in respect of its chemical composition, pH value, and viscosity. If an absorbent/rinsing body activated with the said synthetic activation solution is used, especially effective vacuum therapy of wounds can be performed. This is based on a synergetic interaction between the specially selected synthetic activation solution and the superabsorbent polymer.

In a highly advantageous and therefore especially preferred embodiment, the synthetic activation solution is Ringer's solution.

The advantages of activated absorbent/rinsing bodies in vacuum therapy stated above are obtained to an even greater degree on an absorbent/rinsing body according to the associated dependent claim, which contains Ringer's solution as the activation solution. Ringer's solution is understood to be an iso-osmotic synthetic solution approximating to blood that contains sodium chloride, potassium chloride, and calcium chloride. The Ringer's solution preferably contains 147 mmol/l of sodium ions, 4.0 mmol/l of calcium ions, 3.0 mmol/l of calcium ions, and 157 mmol/l of chloride ions, where an upward or downward deviation of the relevant ion concentration by 5% from the stated value is possible. Ringer's solution can be manufactured, for example, by dissolving 8.6 g of sodium chloride, 0.30 g of potassium chloride, and 0.33 g of calcium chloride in one liter of distilled water. The advantageous effect of Ringer's solution in the vacuum therapy of wounds is especially pronounced in the cleansing phase of wound healing.

The absorbent/rinsing body activated with the synthetic activation solution is preferably provided packaged so that it only needs to be removed from the packaging before the dressing is applied.

It is especially preferred for an absorbent/rinsing body activated with Ringer's solution to be provided packaged so that it only needs to be removed from the packaging before the dressing is applied. It is further especially preferred for the absorbent/rinsing body activated with the synthetic activation solution to be provided packaged in sterile manner.

In a further preferred embodiment, the invention provides a device for the vacuum therapy of wounds, comprising an activated absorbent/rinsing body, where the absorbent/rinsing body is an absorbent/rinsing body of stable volume. The absorbent/rinsing body of stable volume does not increase in volume during the vacuum therapy.

Instead, the absorbent/rinsing body of stable volume reduces in volume during the vacuum therapy. The reduction in volume of the absorbent/rinsing body is accompanied by a reduction in weight of the absorbent/rinsing body.

The volume stability is obtained by activating the absorbent/rinsing body with an aqueous activation solution, preferably with a synthetic activation solution, in particular, with a synthetic activation solution according to the associated dependent claim, especially preferably with Ringer's solution.

It is advantageous for the absorbent/rinsing body of stable volume to contain at least 500 percent by weight, preferably 600 percent by weight, especially preferably 800 percent by weight of the activation solution. In a further advantageous embodiment, the absorbent/rinsing body of stable volume contains at least 1000 percent by weight of the activation solution. The absorbent/rinsing body of stable volume preferably contains less than 5000 percent by weight, especially preferably less than 3500 percent by weight, highly preferably less than 2500 percent by weight of the activation solution.

The at least one superabsorbent polymer contained in the absorbent/rinsing body is at least partially put into the swollen state by the activation.

It has been found that the activated absorbent/rinsing body, if it is exposed to a vacuum of 125 mmHg for 24 h, can remain in a moist, swollen condition, wherein its volume and weight become smaller.

In this context, an absorbent/rinsing body of stable volume is therefore understood to be an activated absorbent/rinsing body that contains at least one superabsorbent polymer, wherein the volume of the activated absorbent/rinsing body reduces at a temperature of 20° C. over 24 h at a vacuum of 125 mmHg by at least 3% and no more than 50%, preferably at least 10% and no more than 35%. A device for measuring the volume stability of the activated absorbent/rinsing body is described in Example 7 of the application.

It has further been found that the absorbent/rinsing body of stable volume can release activation solution during vacuum therapy of wounds and, if applicable, absorb wound exudate in exchange. In this case, the activated absorbent/rinsing body of stable volume typically exhibits a volume reduction during the vacuum therapy in the wound of at least 1% and no more than 50% in relation to the volume at the beginning of the vacuum therapy.

One advantage during use of an absorbent/rinsing body of stable volume in vacuum therapy of wounds is that during the vacuum therapy, even pressure can be maintained constantly. Uneven pressure distribution can feel unpleasant for the patient and impair wound healing. It was discovered that one cause of uneven pressure distribution in the use of conventional absorbent pads could be an increase in volume, in particular, if the increase in volume results in absorbent/rinsing bodies approximating to the circular shape.

The absorbent/rinsing body of stable volume provides a soft wound dressing throughout the entire treatment in vacuum ranges up to 250 mm Hg.

A further advantage when using the absorbent/rinsing body of stable volume in vacuum therapy of wounds is that absorbent/rinsing bodies of stable volume are excellently suitable for padding out the wound space. Because no volume increase of the absorbent/rinsing body occurs during the vacuum therapy, the wound remains evenly padded out during the treatment and no unwanted cavities are formed.

Because of the volume decrease of the absorbent/rinsing body of stable volume during the vacuum therapy, the region near to the wound is relieved of pressure over time. This further positive effect that can occur with the use of the inventive device has proven very advantageous for wound healing.

It has proven especially advantageous that a hydroactive effect can already apply immediately after the beginning of the vacuum therapy if an absorbent/rinsing body of stable volume is used. To study the cause of the hydroactive effect typically starting immediately after the beginning of the treatment, experiments were performed on a wound simulator. A suitable wound simulator is described in DE 102008064510.9. In experiments, it was observed that activation solution can already be released from the activated absorbent/rinsing body immediately after the vacuum dressing is applied. The quantity per unit of time of released activation solution can be very fast, for example, within the first three hours, reach a maximum, and then decline. The quantity and speed of the activation solution released varies depending on parameters such as, in particular, the level of vacuum, but also, for example, on the composition, size, shape, and, if there is one, on the envelope of the activated absorbent/rinsing body. The hydroactive effect can be enhanced in the case of intermittent vacuum setting because the absorbent/rinsing body can release more moisture to the wound during phases of reduced vacuum. In particular, an advantageous rinsing effect can be achieved during use of the absorbent/rinsing body of stable volume by varying the vacuum during the treatment. This permits cleansing of the wound during application.

Experiments on a wound simulator have shown further that a first component of the activation solution released from the activated absorbent/rinsing body is sucked away by the vacuum system and therefore irreversibly removed from the wound space. A second component of the released activation solution, on the other hand, remains at least temporarily in the wound space and could be responsible for the observed hydroactive effect.

A further advantage in the use of an absorbent/rinsing body of stable volume in the vacuum therapy of wounds is that an absorbent/rinsing body of stable volume tends less to adhere to and/or grow together with the base of the wound. This may be because the pressure exerted on the wound base remains constant or reduces during the treatment. This is advantageous because no additional cleaning of the wound is necessary after removal of the vacuum dressing and dressing changing can therefore be performed more gently and faster. Furthermore, traumatization of the wound can be avoided during dressing changing, which increases the effectiveness of the treatment.

Surprisingly, if the absorbent/rinsing body of stable volume is used in vacuum therapy, unobstructed drainage of wound exudate from the device can be better ensured. This advantageous effect may be because the drainage channels in the absorbent/rinsing body or in an additionally present wound contact layer, if there is one, are kept open. With the use of the absorbent pads known from the prior art in which the volume increases during the treatment, on the other hand, drainage channels can be squashed and therefore partially deactivated.

The inventive device comprises a cover material for air-tight closure of the wound. “Air-tight closure” in this context is not understood to be the complete absence of gas exchange between the wound space and its environment. Rather “air-tight closure” in this context is understood to be the degree of closure that is necessary for the vacuum pump used to maintain the vacuum required for the vacuum therapy of wounds. For that reason, cover materials can be used that exhibit slight gas permeability provided that the vacuum that is necessary for the vacuum therapy can be maintained.

The air-tight cover material can be, for example, bowl-shaped and made of solid material or be a flexible film. Combinations of a solid framework or contact plates and flexible films are also conceivable.

In a preferred embodiment of the invention, the cover material for air-tight closure of the wound comprises a water-insoluble polymer, or a metal film.

In an especially preferred embodiment of the invention, the water-insoluble polymer is polyurethane, polyester, polypropylene, polyethylene, polyamide, or polyvinylchloride, polyorganosiloxane (silicone), or a mixture of these.

Further suitable polymer film materials are known to specialists. Finished products having the said properties can also be used as the cover material.

Polyurethane of the brand Hydrofilm® (Paul Hartmann AG, Germany) and Visulin® (Paul Hartmann AG, Germany) have proven to be especially suitable cover material for the inventive device.

The cover material is attached in the region surrounding the wound or at the edge of the wound so that air-tight wound closure is ensured. It can be convenient if the cover material is made self-adhesive over its entire surface or has a self-adhesive edge. Alternatively, it may be attached and sealed, for example, using an adhesive film, a liquid adhesive, or a sealing compound.

In a preferred embodiment of the invention, the cover material comprises a film made of one or more water-insoluble polymers, the film being made self-adhesive over its complete surface or having a self-adhesive edge.

However, it is also possible for the cover material to be held on the wound only by the vacuum produced as part of the vacuum therapy. In a preferred embodiment, the cover material and the means for functional connection of the wound space to a source of vacuum outside the cover material are provided already interconnected and ready to use. It is highly preferred for this embodiment to be a film made of one or more water-insoluble polymers and to have a self-adhesive edge because this configuration makes application of the dressing much easier.

The inventive device for vacuum therapy comprises a means for functional connection of the wound space to a vacuum source located outside the cover material so that a vacuum can be established in the wound space and liquids can be sucked out of the wound space.

In connection with the invention, the expression “vacuum in the wound space” denotes an air pressure that is rendered lower within the wound dressing than the ambient air pressure (atmospheric air pressure).

“Within the wound dressing” refers to the interstice formed between the cover material and the wound. “Vacuum” is frequently also referred to as “negative pressure.”

The pressure difference between the air pressure within the wound dressing and the ambient air pressure is stated in mm Hg (millimeters of mercury) in connection with the invention because this is customary in the field of vacuum therapy. 1 mm Hg corresponds to one Torr or 133.322 Pa (Pascal). In connection with the invention, the vacuum, that is, the pressure difference between the air pressure inside the wound dressing and the ambient air pressure is stated as a positive numerical value in mm Hg.

In one embodiment of the invention, the vacuum is a vacuum of no more than 250 mmHg. This negative pressure range of no more than 250 mmHg has proven suitable for wound healing. In a preferred embodiment of the invention, the vacuum is a vacuum of at least 10 mm Hg and no more than 150 mm Hg.

In two further alternative embodiments, each of them preferred, the vacuum a) is a constant vacuum or b) a time-variable vacuum.

“Constant vacuum” (a) in this case refers to the fact that the vacuum is kept essentially constant during the treatment. “Essentially constant” means that, during the treatment, slight upward or downward changes in the vacuum, for example, by 15%, may occur.

A preferred constant vacuum is the range from at least 80 mm Hg to no more than 150 mm Hg.

“Time-variable vacuum” (b) is, in this case, understood to mean that the vacuum undergoes specific variations during the treatment. Specific variations of the air pressure mean those variations of the air pressure that occur when a first target value for the vacuum has been reached after the vacuum dressing has been applied. By contrast, the initial rise phase of the vacuum that occurs after application of the dressing until the first target value has been reached is not included in the term “time-variable vacuum.” This applies analogously to the drop in the air pressure difference to ambient air pressure that is required at the end of the treatment, which is not covered by the term “time-variable vacuum” either.

The lower limit for the “time-variable vacuum” is the ambient air pressure and the upper limit is a maximum vacuum of 250 mm Hg, preferably 150 mm Hg, in particular, 125 mm Hg. The actual vacuum applied during the treatment varies within this range defined by its limit values. The “time-variable vacuum” therefore includes, for example, a single change or multiple changes between one or more higher vacuum values and one or more lower vacuum values. Similarly, “time-variable vacuum” includes a specific single change or multiple changes occurring during the treatment between the ambient pressure and one or more higher vacuum values.

In a preferred embodiment, the maximum vacuum for a time-variable vacuum is 125 mm Hg. The lower limit for the variation of the vacuum is the ambient air pressure in this embodiment. During the treatment, the vacuum varies between or within these limit values.

In a further preferred embodiment, the maximum vacuum for a time-variable vacuum is 125 mm Hg. The lower limit for the variation of the vacuum in this embodiment is 20 mm Hg. During treatment, the vacuum varies between or within these limit values.

In the two embodiments described above, the change between the upper and the lower pressure value can be periodic or non-periodic. A periodic change is preferred. The time intervals in which the higher vacuum and in which the lower vacuum or ambient pressure is maintained can be of different durations. A lower vacuum is preferably maintained for longer than a higher vacuum. Suitable time intervals in which a certain vacuum setting or the ambient pressure is maintained are, for example, 1 min, 2 min, 5 min, 10 min, 30 min, 1 h, 12 h, or 24 h.

A varying vacuum with the parameters stated below is especially preferred, wherein alternation between the two vacuum values is performed continually at the specified time intervals during the treatment:

A vacuum of 125 mm Hg for 2 min,

thereafter

a vacuum of 20 mm Hg for 5 min.

The inventive device can further comprise a means by which the vacuum actually present in the device can be verified and adjusted, if necessary. The means can be located in the wound space or at another suitable. position. For example, it is possible to attach a pressure sensor in the vacuum tube between the wound dressing and the vacuum source.

The inventive device for vacuum therapy of wounds further comprises a means for the functional connection of the wound space with a source of vacuum located outside the cover material.

The functional connection can be established, for example with a connection tube or with a vacuum connection element. Vacuum connection elements are also known to specialists by the term “port.”

In one embodiment, the means for functional connection of the wound space with a vacuum source located outside the cover material is at least one connecting line. The at least one connecting line can be brought through the cover material.

Alternatively, the at least one connecting line can be routed under the edge of the cover material.

In both cases, the penetration point must be sealed air-tight so that the required vacuum can be maintained in the dressing. An adhesive film, adhesive compound, or adhesive strip is suitable as a sealing means, for example.

The connecting line can, for example, be a tube, a pipe, or another body with a hollow interior. One suitable tube is, for example, a silicone drainage tube.

The connecting line advantageously has a vacuum adapter at the end that is located outside the wound dressing to be connectable with the further components of the vacuum system.

The connecting line has an opening at the end that is located inside the wound dressing.

In a further embodiment, the means for functional connection of the wound space with a vacuum source outside the cover material is a vacuum connection element (port) that can be attached to the inside or outside of the cover material, wherein the cover material has suitable openings. In this embodiment, too, air-tight sealing either of the penetration opening (port inside) or the contact surface (port outside) must be ensured. Sealing can be achieved, for example, with an adhesive film, with an adhesive compound, or with an adhesive strip. It is also conceivable that the port itself has appropriate fastening means such as adhesive surfaces. Suitable vacuum connection elements are commercially available. These are typically vacuum connection elements that are attached to the outside of the cover material.

The vacuum connection element also advantageously has a vacuum adapter to enable connection to the further components of the vacuum system.

In a preferred embodiment of the invention, the device for the vacuum therapy of wounds comprises at least one wound contact layer for inclusion in the interstice that is formed by the activated absorbent/rinsing body and the surface of the wound. The additional wound contact layer can be connected with the activated absorbent/rinsing body adhesively or non-adhesively.

As the wound contact layer, any wound dressing according to the prior art can be used provided that wound exudate can pass through it and the material exhibits no tendency to grow together with or adhere to the wound tissue. The passage of wound exudate can either be ensured by the wound dressing being made of a material that is permeable to liquids. This can, for example, be the case for wound dressings made of foam material. The passage of wound exudate can also be ensured if the wound dressing has suitable openings or channels.

A wound contact layer made of an open-cell or semi-open-cell foam, in particular, polyurethane is especially preferred. The use of wound dressings made of a structured gel, hydrocolloid, or polyorganosiloxane (silicone) is also advantageous.

An especially suitable wound contact layer made of a structured gel is disclosed in the German patent application 102008062472.1. There, a wound dressing for vacuum therapy is described comprising a substrate layer and a wound contact layer applied to the side of the substrate layer facing the wound, wherein the wound contact layer has openings passing through the wound contact layer that are transverse with respect to its surface extent, characterized in that the side of the wound contact layer facing the wound has protuberances and recesses and the wound contact layer forms a contact surface with a skin surface only in the region of the protuberances.

In modified form, the wound dressings described in the German patent applications DE102008031183.9 and DE102008031182.0 can also be used as a wound contact layer for the inventive device. The necessary modification is that, for example, an unobstructed passage of wound exudate is enabled by the inclusion of channel-like openings. The wound contact layers from DE102008031183.9 and DE102008031182.0 are especially suitable for use in the granulation phase and in the epithelization phase. They comprise a hydrophilic polyurethane foam with a water component of at least 10 percent by weight.

Suitable wound dressings made of polyurethane are commercially available as finished products, for example, the commercial product PermaFoam® from Paul Hartmann AG.

Similarly, the wound contact layer can comprise a permeable nonwoven layer or lattice tulle. The permeable nonwoven layer or the tulle preferably consists of a hydrophobic material, for example, polyester. The tulle can further be provided with an ointment.

The ointment compresses Hydrotüll® and Atrauman® (Paul Hartmann AG, Germany) are especially suitable wound contact layers.

The problem of possible growing together or adhesion of the wound dressing to the wound tissue is solved in an alternative embodiment of the invention by having the wound dressing either completely consist of a bioresorbable material or providing it with bioresorbable material on the wound side. Suitable bioresorbable materials are known, for example, from DE19609551 or WO02/072163.

Preferably, in particular for the use of the inventive device in the treatment of infected wounds, the wound dressing has an anti-microbial coating. The anti-microbial coating is preferably a silver coating.

An ointment compress with a silver coating that is suitable as a wound contact layer is the commercial product Atrauman Ag® (Paul Hartmann AG, Germany).

The wound contact layer can contain an antibiotically active pharmaceutical substance.

The wound contact layer can also contain substances that promote wound healing that are released to the surface of the wound during treatment. One example of such substances are growth factors.

In a further embodiment, the inventive device comprises more than one wound contact layer, including layers of different materials. A combination of multiple layers permits optimum adaptation to the particular wound situation. For example, it can be advantageous to use a combination of a silver-coated ointment compress with a foam dressing as the wound contact layer in an infected wound.

In a further preferred embodiment, the device for vacuum therapy of wounds comprises at least one additional pressure distribution layer for inclusion in the interstice that is formed by the activated absorbent/rinsing body and the cover material.

The at least one additional pressure distribution layer is independent of the wound contact layer stated above.

The advantage of an additional pressure distribution layer can be that the pressure exerted on the base of the wound by the dressing is distributed more evenly by use of the pressure distribution layer. Furthermore, the pressure distribution layer can store additional wound exudate.

The additional pressure distribution layer can consist of an open-cell or semi-open-cell foam, a spacer knitted fabric, a textile layer, a structured gel, or a permeable nonwoven layer. Folded-edge compresses or lattice tulles are suitable textile layers.

The additional pressure distribution layer can be constituted such that liquid such as wound exudate is allowed to pass through it. The pressure distribution layer can contain suitable channels or openings or be made of a material that is permeable to liquids.

The additional pressure distribution layer can be connected adhesively or non-adhesively with the activated absorbent/rinsing body. It can also be separated from the absorbent/rinsing body by a further layer, for example, one sheet of a textile layer.

Further, the invention provides a ready-to-use set for vacuum wound therapy comprising an air-impermeable cover material for air-tight closure of the wound and the wound environment, a means for the functional connection of the wound space to a source of vacuum located outside the cover material so that a vacuum can be established in the wound space and liquids can be sucked out of the wound space, and at least one activated absorbent/rinsing body that contains at least one superabsorbent polymer, for inclusion in the interstice that is formed by the wound surface and the cover material, wherein the activated absorbent/rinsing body is provided packaged and ready to use.

In a preferred embodiment, the set comprises cover material, a means for the functional connection of the wound space to a source of vacuum located outside the cover material, and at least one activated absorbent/rinsing body that is packaged and ready to use according to the associated dependent claims.

The absorbent/rinsing body that is part of the set can be produced by activating the dry absorbent/rinsing body by wetting it with an activation solution, preferably, Ringer's solution, and packaging it in a liquid-tight package. The activated and ready-to-use packaged absorbent/rinsing body is preferably provided in a sterile form. Ideally, the activated absorbent/rinsing body is provided in a sterile, individually sealed, ready-to-use package. Sterilization can be performed by counterpressure autoclaving or other sterilization methods known to specialists to be suitable.

The set can contain further optional components, such as, for example, one or more wound contact layers, one or more additional pressure distribution layers, adhesion means for affixing the dressing, sealing means for achieving air-impermeable sealing of the dressing, pressure sensors, connecting elements for pressure sensors, additional tubes, connecting elements for tubes, disinfection means, skincare means, pharmaceutical preparations, or instructions for use.

The air-impermeable cover material is preferably a cover material according to the associated dependent claims.

In a preferred embodiment, the set includes at least one wound contact layer according to the associated dependent claims.

In a further preferred embodiment, the set comprises at least one additional pressure distribution layer according to any one of the associated dependent claims.

The set can also include both at least one wound contact layer and at least one additional pressure distribution layer.

This set preferably further comprises a ready to use vacuum unit. The vacuum unit can contain components such as a pump, one or more liquid vessels, a control unit, a power supply unit, electrical connection means, and tubes. The vacuum unit can also contain a device for the functional connection of the vacuum dressing to an existing stationary source of vacuum.

All components for which it is medically necessary are packaged in a sterile manner. In particular, the activated absorbent/rinsing body, the cover material, the means for functional connection of the wound space with a source of vacuum located outside the cover material, and the optionally provided wound dressing or additional pressure distribution layer are preferably provided packaged in a sterile manner.

The advantage of the ready to use set is that the vacuum dressing can be applied in a standardized and uncomplicated manner. A further advantage is that all components of the set used in the region of the wound can already be provided sterilized.

In a further especially preferred embodiment, an activated absorbent/rinsing body that contains at least one superabsorbent polymer is provided for use in the vacuum therapy of wounds. The activated absorbent/rinsing body can be used in vacuum therapy devices for the treatment of wounds. Special advantages arise if the wounds are burns, wounds produced by mechanical traumatization, a wound produced by the effect of chemicals, wounds caused by a metabolic disorder, a wound caused by a circulatory disorder, or a wound caused by a pressure ulcer. The activated absorbent/rinsing body as a component of a vacuum therapy device permits fast, effective, and gentle treatment of wounds. The advantages of the activated absorbent/rinsing body are based, among other characteristics, on a soft structure, on the volume stability, on the reduced tendency to adhesion to and/or growing together with the base of the wound, and on the high absorption capacity.

In a further preferred embodiment, an activated absorbent/rinsing body that contains at least one superabsorbent polymer is provided for use in vacuum therapy in the treatment of a wound produced by a skin grafting. The application comprises the treatment of wounds produced by split-thickness grafts and by full-thickness grafts by means of vacuum therapy. Advantageous effects result due to the soft structure of the activated absorbent/rinsing body, due to the even pressure distribution, and due to the hydroactive effect. If the activated absorbent/rinsing body is used to treat a wound produced by skin grafting, the skin graft can be sufficiently fixed while avoiding harmful shearing forces.

A further object of the invention is a method for vacuum therapy of wounds comprising the steps

a) Provision of a device or of a set according to the independent claims

b) Application of the vacuum dressing to the wound

c) Production of a vacuum of no more than 250 mm Hg in the wound space for at least 30 minutes and no longer than 7 days.

Below the inventive device for the vacuum therapy of wounds is explained in more detail using drawings. However, the invention is not to be understood as reduced to the embodiments illustrated in the drawings or in the description of the drawings. Rather the inventive device also comprises combinations of the individual characteristics of the alternative forms.

### BRIEF DESCRIPTION OF THE DRAWING

FIG. 1 schematic structure of the inventive device for vacuum therapy of wounds (side view);

FIG. 2 schematic structure of an embodiment (side view);

FIG. 3 schematic structure of a further embodiment (side view);

FIG. 4 schematic structure of a further embodiment (side view);

FIG. 5 schematic structure of a further embodiment (side view);

FIG. 6 schematic structure of a further embodiment (side view);

FIG. 7 schematic structure of a further embodiment (side view); and

FIG. 8 schematic structure of an experimental set-up for determining the volume stability of activated absorbent/rinsing bodies under a vacuum (side view).

### DESCRIPTION OF THE PREFERRED EMBODIMENT

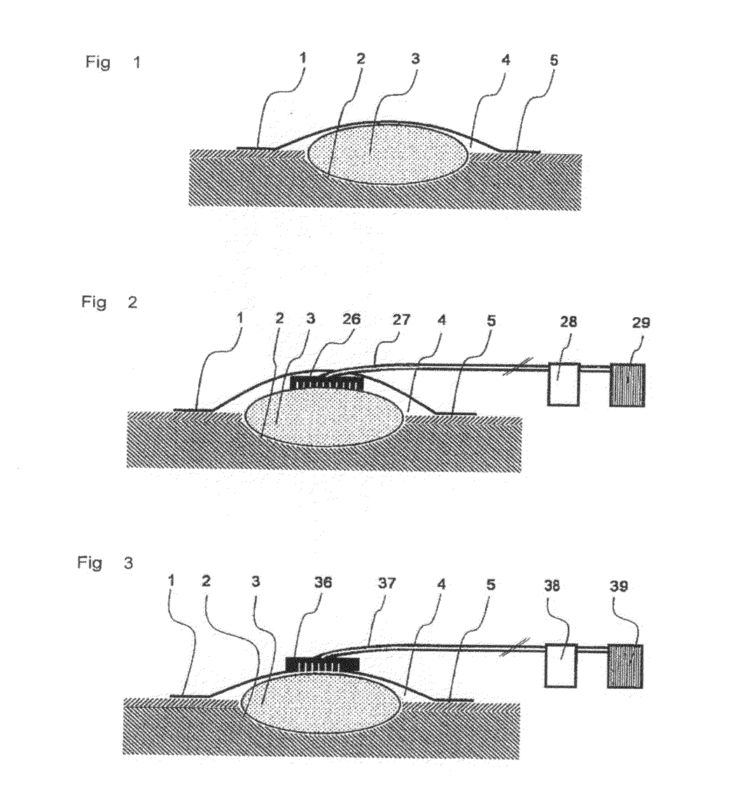

FIG. 1 shows the schematic structure of the inventive device in a side view. The device comprises an air-impermeable cover material (1), a means (not depicted in FIG. 1) for functional connection of the wound space (4) to a source of vacuum located outside the cover material (not depicted in FIG. 1) and at least one activated absorbent/rinsing body (3) that contains at least one superabsorbent polymer. The cover material (1) is attached in the region surrounding the wound (5), which usually has undamaged skin. The size of the cover material should be such that the cover material can be attached outside the wound region in the region surrounding the wound (5). The cover material (1) can have different dimensions and shapes, for example, circular, oval, or rectangular. It can also be provided in an irregular shape fitted to the wound. The cover material (1) can be an opaque material, a partially transparent material, or a completely transparent material. The use of transparent material can be advantageous for inspecting the progress of wound healing. The use of only partially transparent or opaque material can be advantageous in that it spares the patient the sight of the wound. Alternatively, the cover material (1) can also be a rigid material that is applied over the wound region in the form of a bowl that is open toward the wound and is attached in the region surrounding the wound (5). The cover material (1) must be attached in the region surrounding the wound (5) and sealed air-tight. This can be achieved, for example, if the cover material (1) has an adhesive edge. The adhesive edge should be protected to the greatest extent possible by protective strips until the dressing is applied. Alternatively, an adhesive substance can be applied either to the edge of the cover material (1) and/or to the undamaged skin in the region surrounding the wound. This has the advantage that adaptation of the cover material to the shape and size of the wound is easier. Attachment and air-tight sealing of the device can also be achieved by the use of adhesive strips or an adhesive compound.

FIG. 1 further shows an activated absorbent/rinsing body (3). When the dressing is applied, the activated absorbent/rinsing body (3) should be inserted into the wound space (4) in such a way that as few cavities as possible result and good contact with the base of the wound is ensured (2). For this purpose, one or more activated absorbent/rinsing bodies of a suitable size should be chosen.

FIG. 2 shows the schematic structure of an embodiment of the inventive device in a side view. The device comprises an air-impermeable cover material (1), a vacuum connection element (26) for the functional connection of the wound space (4) by means of a tube (27) with a vacuum unit (29) located outside the cover material, and at least one activated absorbent/rinsing body (3) that contains at least one superabsorbent polymer. In the embodiment shown here, the vacuum connection element (26), which is also termed “port,” is located on the inside of the air-impermeable cover material (1) facing toward the wound. Between the vacuum connection element (26) and the vacuum unit (29) there is a collection vessel (28). In this embodiment, sealing and adhesive attachment must be ensured between the top of the port and the cover material. The end of the port facing away from the wound typically extends through the cover material and is connected with the vacuum system.

The schematic structure of a further embodiment of the inventive device is shown in a side view in FIG. 3. The device differs from the device shown in FIG. 2 in that, in the embodiment shown here, the vacuum connection element (36) is located on the outside of the air-impermeable cover material (1) facing away from the wound. To connect the wound space (4) functionally to a vacuum unit (39) located outside the cover material, in this configuration, one or more openings passing through the cover material (1) must be located in the region of the vacuum connection element (36). Furthermore, air-tight sealing must be ensured. Such sealing can be achieved, for example, by applying a film (not depicted in FIG. 3) that is adhesively attached to the cover material (1) to the top of the port facing away from the wound. Applying the dressing can be made easier by using a port in which a suitable attachment and sealing means is provided to attach the port on the cover material. This is, for example, the case for the commercially available PPM-Drainageport® from Phametra-Pharma and Medica-Trading GmbH (Herne/Ruhrstadt, Germany).

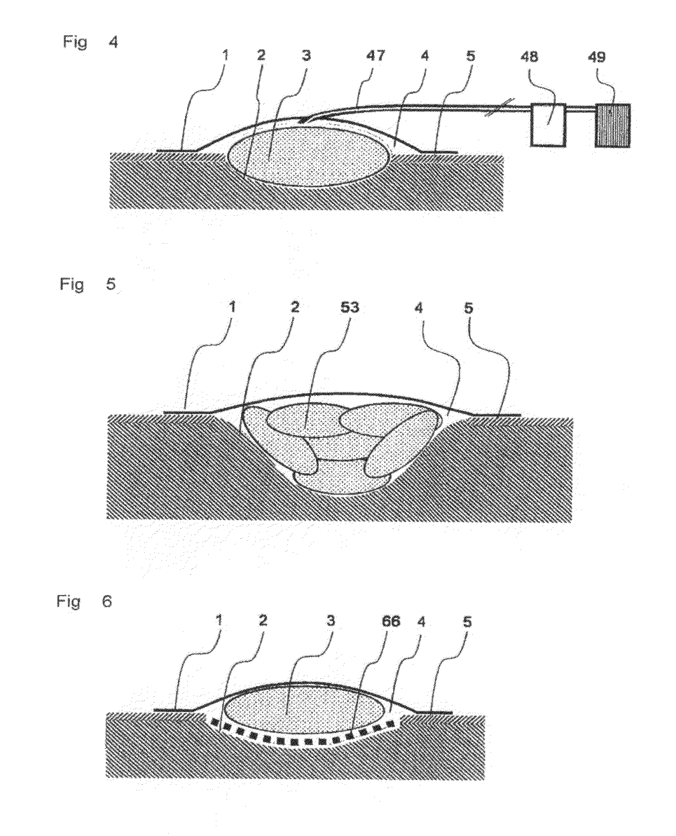

FIG. 4 shows the schematic structure of a further embodiment of the inventive device in a side view. In this embodiment, use of a port is dispensed with. Instead, the functional connection between the wound space (4) and the vacuum unit (49) located outside the cover material is established by a tube (47) passed through the cover material (1). Alternatively, the tube can be passed under the edge of the cover material (not depicted). Appropriate air-tight sealing must be ensured. The tube has one or more openings at the end located in the wound space. The use of a tube with a sieve-like end element having multiple openings (not depicted) can be advantageous because blockage of the tube can thus be avoided.

FIG. 5 shows the schematic structure of a further alternative embodiment of the inventive device in a side view. The device comprises an air-impermeable cover material (1), a means for functional connection of the wound space (4) to a source of vacuum (not depicted) located outside the cover material, and more than one activated absorbent/rinsing body (53) containing at least one superabsorbent polymer. This embodiment is especially suitable for deep or creviced wounds. By using multiple activated absorbent/rinsing bodies (53) of a suitable size, a very good fit to the shape of the wound can be achieved. The means (not depicted) for functional connection of the wound space (4) to a source of vacuum located outside the cover material (1) can, for example, be implemented as shown in FIGS. 2 to 4.

The use of a wound contact layer (66) in the inventive device is shown by way of example in FIG. 6 in a side view. This is a preferred embodiment of the inventive device. The device comprises an air-impermeable cover material (1), a means (not depicted) for the functional connection of the wound space (4) to a source of vacuum (not depicted) located outside the cover material, at least one activated absorbent/rinsing body (3) that contains at least one superabsorbent polymer and a wound contact layer (66) in the interstice formed by the activated absorbent/rinsing body (3) and the wound surface (2). The wound contact layer (66) should be constituted such that wound exudate can pass through the wound contact layer (66). It is also possible to use multiple wound contact layers. In this way, differing wound contact layers can be combined. The wound contact layer (66) can also comprise fibers or tamponade strips.

The additional wound contact layer (66) can be connected adhesively or non-adhesively with the activated absorbent/rinsing body. It can also be separated from the absorbent/rinsing body by a further layer, for example, one sheet of a textile layer.

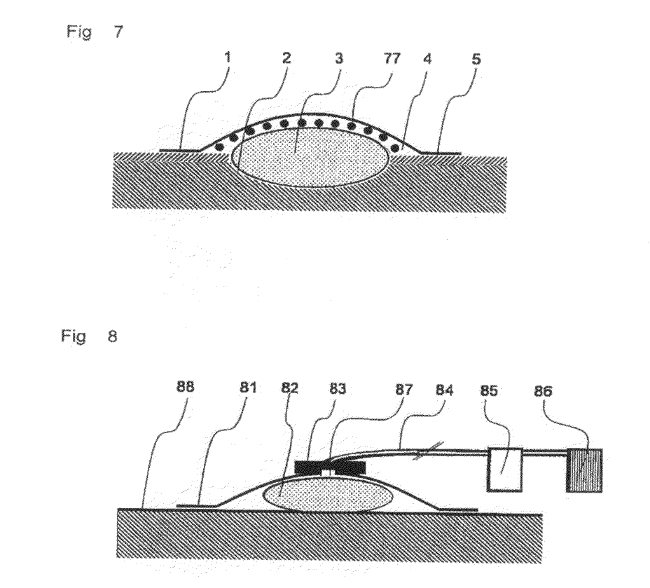

FIG. 7 shows the schematic structure of a further preferred embodiment of the inventive device in a side view. This embodiment is characterized by the presence of an additional pressure distribution layer (77). The device therefore comprises an air-impermeable cover material (1), a means (not depicted) for functional connection of the wound space (4) to a source of vacuum (not depicted) located outside the cover material, at least one activated absorbent/rinsing body (3) that contains at least one superabsorbent polymer and a pressure distribution layer (77) in the interstice formed by the activated absorbent/rinsing body (3) and the air-impermeable cover material (1). It must be emphasized that the invention also comprises a combination of the wound contact layer shown in FIG. 6 with the additional pressure distribution layer shown in FIG. 7 and that this is an especially preferred embodiment.

It is also possible to use multiple pressure distribution layers, in which case different pressure distribution layers can be combined. (The embodiments that comprise multiple pressure distribution layers are not shown.)

The additional pressure distribution layer (77) can be constituted such that liquid, such as wound exudate, is allowed to pass through it. For this purpose, the pressure distribution layer can contain suitable channels or openings. Alternatively, it can consist of a material that allows the passage of wound exudate without further preparation.

The additional pressure distribution layer (77) can be constituted such that it is attuned to the means for the functional connection of the wound space (4) with a source of vacuum located outside the cover material. Such an adaptation could, for example, be that the pressure distribution layer(s) provides a hollow for the insertion of the end of the tube or the port. Other adaptations are conceivable.

The additional pressure distribution layer can be connected adhesively or non-adhesively with the activated absorbent/rinsing body. It can also be separated from the absorbent/rinsing body by a further layer, for example, one sheet of a textile layer.

FIG. 8 shows the schematic structure of an experimental set-up for determining the volume stability of activated absorbent/rinsing bodies under a vacuum in a side view. The experimental set-up is located on a plate (88) with a smooth, water-resistant surface and comprises an air-impermeable cover material (81), an activated absorbent/rinsing body (82), a vacuum connection element (83) that can be attached to the outside of the cover material, wherein the cover material has suitable openings (87), a connecting line (84), a collection vessel (85), and a vacuum unit (86). The vacuum connection element (83) is located on the outside of the air-impermeable cover material (81) facing away from the plate.

To measure the volume stability of an activated absorbent/rinsing body under a vacuum, first the weight m₁ and the volume V₁ of the activated absorbent/rinsing body are determined. To obtain the volume of the absorbent/rinsing body, a glass beaker is filled with distilled water, the absorbent/rinsing body is placed in it, and the displaced volume is collected using a graduated cylinder. The absorbent/rinsing body must be enveloped in a thin plastic film for this operation to prevent liquid from being absorbed during volume measurement. The film is removed again immediately after the volume has been measured. Then the activated absorbent/rinsing body is placed on the plate (88). The absorbent/rinsing body (82) is then completely covered by an air-impermeable cover material (81) wherein the edge of the cover material is adhered to the plate in an air-tight fashion. The cover material is preferably a film that has a self-adhesive surface at least at the edge. Alternatively, the cover material can, for example, be attached to the plate in an air-tight fashion using an adhesive strip. Then a hole measuring approx. 0.5 cm in diameter is cut into the center of the cover material above the absorbent/rinsing body. On the outside of the film, a vacuum connection element (83) is then attached in the region of the hole and adhered to the cover material in an air-tight fashion. The vacuum connection element is connected to a collection vessel (85) and to a vacuum unit (86) by means of a connecting line (84) in such a way that a vacuum can be produced in the space formed between the cover material (81) and the plate (88). Then a vacuum of 125 mm Hg is applied for 24 h, ensuring that the measuring apparatus is maintained at a temperature of 20° C. (±3° C.). During the experiment, no additional liquid is supplied to the activated absorbent/rinsing body from outside. After 24 h have elapsed, the vacuum connection element and the cover material are removed and the final weight m₂ and the final volume V₂ of the absorbent/rinsing body are measured as described above. The absolute weight change Am during the experiment results from the difference m₁−m₂. The relative weight change Δmᵣ as a percentage of the initial weight is calculated by (((m₁−m₂): m₁)×100). The absolute volume change ΔV during the experiment in relation to the initial volume results from the difference V₁−V₂. The relative volume change ΔVᵣ as a percentage of the initial volume is calculated by (((V₁−V₂): V₁)×100).

### Examples

### Example 1

Vacuum therapy of a wound is performed on a patient by first inserting a sterile activated absorbent/rinsing body (TenderWet®, Paul Hartmann AG, Germany) of a suitable size into the wound space. Thereafter, the wound is closed air-tight with a cover film (Hydrofilm®, Paul Hartmann AG, Germany). The self-adhesive cover film is attached on the undamaged skin in the region immediately surrounding the wound. A hole measuring approx. 0.5 cm is cut into the center of the cover film. A PPM-Drainageport® (Phametra-Pharma and Medica-Trading GmbH) is placed over the hole and attached in an air-tight fashion. The drainage port is connected to the vacuum system by means of a tube. A constant vacuum of 125 mm Hg is applied. The dressing is changed and the progress of healing is inspected after three days.

### Example 2

Vacuum therapy of a wound is performed as described in Example 1 except that, before insertion of the absorbent/rinsing body, a layer of Atrauman Ag® (Paul Hartmann AG, Germany) is applied directly to the base of the wound as a wound contact layer.

### Example 3

Vacuum therapy of a wound is performed as described in Example 2 except that, before insertion of the absorbent/rinsing body, a layer of Hydrotüll® (Paul Hartmann AG, Germany) is applied directly to the base of the wound as a wound contact layer.

### Example 4

Vacuum therapy of a wound is performed as described in Example 2 except that, before attachment of the air-tight cover film, a folded-edge compress (ES-Kompresse, Paul Hartmann AG, Germany) is placed directly on the absorbent/rinsing body as an additional pressure distribution layer.

### Example 5

Different embodiments of the inventive device were tested on the wound simulator described in DE 102008064510.9. Table 1 shows the set-up of the vacuum device. The columns therein mean:

a: Cover material for air-tight closure of the wound

b: Means for the functional connection of the wound space with a source of vacuum located outside the cover material

c: Optional wound contact layer, if used

d: Optional additional pressure distribution layer, if used

In the experiments stated below, a TenderWet® (Paul Hartmann AG, Germany) absorbent/rinsing body (round, 5.5 cm in diameter) activated with 15 ml of Ringer's solution was used as the absorbent/rinsing body. The experimental set-ups were each tested with a constant vacuum of 125 mm Hg for three days (72 h) or with a varying vacuum (vacuum of 125 mm Hg for 2 min, then a vacuum of 20 mm Hg for 5 min) for three days (72 h). The wound simulator was adjusted in such a way that, for 72 h, a total of 350 ml of blood substitute (30° C.) were supplied to the artificial wound. The blood substitute sucked out of the artificial wound was collected in a collection vessel. The blood substitute was obtained by adding 9 g of NaCl and 0.2 g of the colorant “Allura Red” to a mixture of 425 g of glycerol and 566 g of demineralized water.

| No. | a | b | c | d |
|---|---|---|---|---|
| 1 | Hydrofilm ® | PPM- | — | — |
| | transparent | Drainageport ® | | |
| | PU film (Paul | (Phametra- | | |
| | Hartmann | Pharma and | | |
| | AG) | Medica-Trading | | |
| | | GmbH) attached | | |
| | | on top of cover | | |
| | | film | | |
| 2 | Hydrofilm ® | PPM- | Atrauman ® Ag | — |
| | | Drainageport ® | silver-containing | |
| | | attached on top | ointment dressing | |
| | | of cover film | (Paul Hartmann | |
| | | | AG) | |
| 3 | Hydrofilm ® | PPM- | — | ES-Kompresse |
| | | Drainageport ® | | folded-edge |
| | | attached on top | | gauze |
| | | of cover film | | compress (Paul |
| | | | | Hartmann AG) |
| 4 | Hydrofilm ® | PPM- | Atrauman ® Ag | ES-Kompresse |
| | | Drainageport ® | | |
| | | attached on top | | |
| | | of cover film | | |
| 5 | Hydrofilm ® | PPM- | Hydrotüll ® | — |
| | | Drainageport ® | hydroactive | |
| | | attached on top | ointment | |
| | | of cover film | compress (Paul | |
| | | | Hartmann AG) | |
| 6 | Hydrofilm ® | PPM- | Hydrotüll ® | ES-Kompresse |
| | | Drainageport ® | | |
| | | attached on top | | |
| | | of cover film | | |
| 7 | Hydrofilm ® | PPM- | PermaFoam ® | — |
| | | Drainageport ® | hydroactive PU | |
| | | attached on top | foam dressing | |
| | | of cover film | (Paul Hartmann | |
| | | | AG) | |
| 8 | Hydrofilm ® | PPM- | PermaFoam ® | PermaFoam ® |
| | | Drainageport ® | cavity | cavity |
| | | attached on top | | |
| | | of cover film | | |
| 9 | Hydrofilm ® | PPM- | — | PermaFoam ® |
| | | Drainageport ® | | cavity |
| | | attached on top | | |
| | | of cover film | | |
| 10 | Hydrofilm ® | PPM- | PermaFoam ® | ES-Kompresse |
| | | Drainageport ® | cavity | |
| | | attached on top | | |
| | | of cover film | | |
| 11 | Hydrofilm ® | PPM- | — | Zetuvit ®- |
| | | Drainageport ® | | Kompresse |
| | | attached on top | | absorbent |
| | | of cover film | | compress (Paul |
| | | | | Hartmann AG) |
| 12 | Hydrofilm ® | PPM- | — | Medicomp ®- |
| | | Drainageport ® | | Kompresse |
| | | attached on top | | nonwoven |
| | | of cover film | | compress (Paul |
| | | | | Hartmann AG) |
| 13 | Hydrofilm ® | PPM- | — | — |
| | | Drainageport ® | | |
| | | attached under | | |
| | | cover film | | |
| 14 | Hydrofilm ® | Silicone tube | — | — |
| | | brought through | | |
| | | the cover film | | |
| 15 | Hydrofilm ® | Silicone tube | Hydrotüll ® | — |
| | | brought through | | |
| | | the cover film | | |
| 16 | Hydrofilm ® | Silicone tube | — | — |
| | | the brought | | |
| | | under edge of | | |
| | | the cover film | | |
| 17 | Tegaderm ™ | PPM- | — | — |
| | Film | Drainageport ® | | |
| | transparent | attached on top | | |
| | film | of cover film | | |
| | (3 M ™) | | | |
| 18 | Visulin ® | PPM- | — | — |
| | transparent | Drainageport ® | | |
| | PU film (Paul | attached on top | | |
| | Hartmann | of cover film | | |
| | AG) | | | |
| 19 | Hydrofilm ® | PPM- | Mepitel ® | |
| | | Drainageport ® | silicone | |
| | | attached on top | net dressing | |
| | | of cover film | (Mölnlycke | |
| | | | Health | |
| | | | Care GmbH) | |
| 21 | Hydrofilm ® | PPM- | PPM- | |
| | | Drainageport ® | Wundschaum | |
| | | attached on top | large-pored PU | |
| | | of cover film | foam (Pharmetra- | |
| | | | Pharma und | |
| | | | Medica-Trading | |
| | | | GmbH) | |
| 22 | Hydrofilm ® | PPM- | PPM- | |
| | | Drainageport ® | Wundschaum | |
| | | attached on top | small-pored PU | |
| | | of cover film | foam (Pharmetra- | |
| | | | Pharma und | |
| | | | Medica-Trading | |
| | | | GmbH) | |
| 23 | Hydrofilm ® | PPM- | PPM- | PPM- |
| | | Drainageport ® | Wundschaum | Wundschaum |
| | | attached on top | large-pored PU | large-pored |
| | | of cover film | foam | PU foam |

### Example 6

In an experiment according to No. 5 from Example 5, the initial weight m₁ and the final weight m₂ after 72 h of the TenderWet Active® absorbent/rinsing body were determined. During the experiment, a constant vacuum of 125 mm Hg was maintained.

The initial weight m₁ was 18.6 g, the final weight m₂ was 14.3 g. This corresponds to a relative weight change Δmᵣ of 23%.

### Example 7

The volume stability of an inventive absorbent/rinsing body (Tenderwet®; 7.5 cm×7.5 cm, dry weight before activation 3.48 g) activated with 40 ml of Ringer's solution was measured under a vacuum of 125 mm Hg, wherein the measuring apparatus was kept at approx. 20° C. during the experiment. During the experiment, no additional liquid was supplied to the activated absorbent/rinsing body from outside. A self-adhesive Hydrofilm® film (Paul Hartmann AG, Germany) was used as the cover material. Furthermore, a PPM-Drainageport® (Phametra-Pharma and Medica-Trading GmbH) was used as the vacuum connection element. An Atmos S04 pump (Atmos MedizinTechnik GmbH & Co. KG, Lenzkirch, Germany) was used as the source of vacuum.

The activated TenderWet® absorbent/rinsing body had an initial weight m₁ of 43.46 g and an initial volume V₁ of 41 cm³. After 24 h of vacuum, the absorbent/rinsing body had a final weight m₂ of 25.06 g and a final volume V₂ of 32 cm³.

This corresponds to a relative weight change of Δmᵣ of 42.34% and a relative volume change ΔVᵣ of 21.95%.

The activated absorbent/rinsing body has a moist and very soft surface at the beginning of the experiment. This property was retained over the duration of the experiment (24 hours).

### LIST OF REFERENCE SYMBOLS

1 Air-impermeable cover material

2 Wound surface or wound base

3/53 Activated absorbent/rinsing body that contains at least one superabsorbent polymer

4 Wound space

5 Wound environment (i.e. usually undamaged skin)

26/36 Vacuum connection element (port)

27/37/47 Connecting line

28/38/48 Collection vessel

29/39/49 Vacuum unit

66 Wound contact layer

77 Pressure distribution layer

81 Air-impermeable cover material

82 Activated absorbent/rinsing body that contains at least one superabsorbent polymer

83 Vacuum connection element (port)

84 Connecting line

85 Collection vessel

86 Vacuum unit

87 Opening in the air-impermeable cover material

88 Plate with smooth surface

We claim:

1. A device for vacuum therapy of a wound, the device comprising:
an air-impermeable cover material for air-tight closure of the wound and of a wound environment;
an element for functional connection of a wound space to a source of vacuum located outside the cover material so that a vacuum can be established in the wound space to suction liquids out of the wound space; and
at least one activated absorbent/rinsing body that contains at least one superabsorbent polymer for insertion in an interstice formed between a wound surface and the cover material.

2. The device for vacuum therapy of wounds of claim 1, wherein the superabsorbent polymer consists of particles or fibers.

3. The device for vacuum therapy of wounds of claim 1, wherein the superabsorbent polymer is a superabsorbent polyacrylate.

4. The device for vacuum therapy of wounds of claim 1, wherein the superabsorbent polymer is a particle mixture of polyacrylate particles of different sizes, the particle mixture containing 5 to 100 percent by weight of particles with a particle size x, wherein x≦300 μm and 0 to 95 percent by weight of particles with a particle size x wherein x>300 μm.

5. The device for vacuum therapy of wounds of claim 1, wherein the activated absorbent/rinsing body is an absorbent/rinsing body surrounded by a textile envelope.

6. The device for vacuum therapy of wounds of claim 1, wherein the activated absorbent/rinsing body is an enveloped air-laid that comprises a superabsorbent polyacrylate, cellulose fibers, and polypropylene fibers.

7. The device for vacuum therapy of wounds of claim 1, wherein the activated absorbent/rinsing body contains at least 500 percent by weight, relative to a weight of dry absorbent/rinsing body, of an aqueous activation solution, the aqueous activations solution comprising more than 50 percent by volume of water and at least 5 mmol/l of sodium ions, wherein the activation solution is a synthetic activation solution.

8. The device for vacuum therapy of wounds of claim 7, wherein the activated absorbent/rinsing body contains at least 500 percent by weight, relative to a weight of dry absorbent/rinsing body, of an aqueous activation solution, the aqueous activation solution having more than 50 percent by volume of water, at least 5 mol/l of sodium ions, at least 0.1 mmol/l of potassium ions, at least 0.1 mmol/l of calcium ions, at least 5 mmol/l of chloride ions, a pH value 4 to 9 and a viscosity at 20° C. between 0.8 mPa s and 150 mPa s.

9. The device for vacuum therapy of wounds of claim 8, wherein the aqueous activation solution has at least one of further inorganic cations and/or anions, organic anions, antimicrobial substances and added bioorganic compounds.

10. The device for vacuum therapy of wounds of claim 8, wherein the activation solution is Ringer's solution.

11. The device for vacuum therapy of wounds of claim 1, wherein the activated absorbent/rinsing body is an absorbent/rinsing body of stable volume.

12. The device for vacuum therapy of wounds of claim 1, wherein the activated absorbent/rinsing body exhibits a reduction in volume during wound treatment relative to a volume at a beginning of the vacuum therapy of at least 1% and no more than 50%.

13. The device for vacuum therapy of wounds of claim 1, wherein the element for functional connection of the wound space to a source of vacuum located outside the cover material is at least one connecting line passing through the cover material at least one connecting line passing under the edge of the cover material or a vacuum connection element that can be attached on an inside or an outside of the cover material, wherein the cover material has suitable openings.

14. The device for vacuum therapy of wounds of claim 1, wherein the vacuum is a vacuum of no more than 250 mmHg.

15. The device for vacuum therapy of wounds of claim 14, wherein the vacuum is at least 10 mm Hg and no more than 150 mm Hg.

16. The device for vacuum therapy of wounds of claim 1, wherein the vacuum is a constant vacuum or a vacuum varying over time.

17. The device for vacuum therapy of wounds of claim 1, wherein the cover material is a film comprising a water-insoluble polymer or a metal film.

18. The device for vacuum therapy of wounds of claim 17, wherein the water-insoluble polymer is polyurethane, polyester, polypropylene, polyethylene, polyamide, polyvinylchloride, polyorganosiloxane or a mixture of these.

19. The device for vacuum therapy of wounds of claim 1, further comprising at least one wound contact layer for inclusion in the interstice formed by the activated absorbent/rinsing body and the surface of the wound.

20. The device for vacuum therapy of wounds of claim 19, wherein the wound contact layer is a polyurethane layer, a hydrocolloid layer, a structured gel, a polyorganosiloxane layer, a permeable nonwoven layer or a lattice tulle.

21. The device for vacuum therapy of wounds of claim 1, further comprising at least one additional pressure distribution layer for inclusion in the interstice formed by the activated absorbent/rinsing body and the cover material.

22. The device for vacuum therapy of wounds of claim 21, wherein the pressure distribution layer is an open-cell or semi-open-cell foam, a spacer knitted fabric, a textile layer, a structured gel or a permeable nonwoven layer.

23. The device for vacuum therapy of wounds of claim 22, wherein the textile layer is a textile wound compress or a lattice tulle.

24. A ready-to-use set for vacuum therapy of a wound, the set comprising:
an air-impermeable cover material for air-tight closure of the wound and a wound environment;
an element for functional connection of a wound space to a source of vacuum located outside the cover material so that a vacuum can be established in the wound space to suction liquids out of the wound space; and
at least one activated absorbent/rinsing body that contains at least one superabsorbent polymer for inclusion in an interstice formed by a wound surface and the cover material, wherein the activated absorbent/rinsing body is provided packaged, ready for use.

25. An activated absorbent/rinsing body that contains at least one superabsorbent polymer for use in the vacuum therapy of wounds.

26. The activated absorbent/rinsing body of claim 25, wherein the wound is a burn, a wound produced by mechanical traumatization, a wound produced by the effect of chemicals, a wound caused by a metabolic disorder, a wound caused by a circulatory disorder or a wound caused by a pressure ulcer.
